(19) United States
(12) Patent Application Publication
(10) Pub. No.: US 2010/0137458 A1
(43) Pub. Date: Jun. 3, 2010

(54) GAS TO LIQUIDS PLANT WITH CONSECUTIVE FISCHER-TROPSCH REACTORS AND HYDROGEN MAKE-UP

(75) Inventor: Rytter Erling, Trondheim (NO)

Correspondence Address: PATTERSON THUENTE CHRISTENSEN PEDERSEN, P.A., 4800 IDS CENTER, 80 SOUTH 8TH STREET, MINNEAPOLIS, MN, 55402-2100, US

(73) Assignee: GTL.F1 AG, Zurich (CH)

(21) Appl. No.: 12/515,933

(22) PCT Filed: Nov. 23, 2007
(86) PCT No.: PCT/GB07/04484

§ 371 Date: Feb. 1, 2010

(30) Foreign Application Priority Data
Nov. 23, 2006 (GB) 0623394.4

(51) Int. Cl. C07C 27/06

(52) U.S. Cl. 518/702; 518/706; 518/705

(57) ABSTRACT

A process for converting synthesis gas to hydrocarbons, using a Fischer-Tropsch synthesis. Two F-T reactors are used in series with water removal between them and additional hydrogen added to the second reactor in an embodiment.

### PRIORITY CLAIM

The present application is a National Phase entry of PCT Application No. PCT/GB2007/004484, filed Nov. 23, 2007, which claims priority from Great Britain Application Number 0623394.4, filed Nov. 23, 2006, the disclosures of which are hereby incorporated by reference herein in their entirety.

### BACKGROUND

In the production of hydrocarbons by the Fischer-Tropsch (FT) process there are several options as to feedstock, including natural gas, coal, heavy oil, biomass etc. Further, a number of products can be synthesized as primary or secondary products, e.g. wax, diesel fuel, olefins, base oil, petrochemical naphtha etc. Common to these variations is that synthesis gas is produced first, and this syngas is then converted by a FT-type polymerization reaction.

There are many syngas technologies and combinations, but one attractive option today for natural gas (NG) feed is to use autothermal reforming (ATR) coupled with a prereformer and an air separation unit (ASU). It is also known that there is at present considerable interest in commercializing this technology based on NG in mega-plants with a size of 60,000 bbl/d or larger of the main products diesel and naphtha. Two medium size plants converting natural gas exist, but these are not considered to employ the most efficient technology.

The main challenges for all FT-plants as well as for the new mega-plants are reduction of investment per barrel product and high carbon efficiency, i.e. reduction in CO₂ emission. In addition, there can also be limitations to feasible FT-reactor sizes not only from a technical and manufacturing point of view, but also because of transport to and assembly in remote areas.

### SUMMARY

According to an embodiment, therefore, there is provided a process for converting synthesis gas comprising hydrogen and carbon monoxide into hydrocarbons using a Fischer-Tropsch synthesis reaction, which comprises conveying a gas feed of hydrogen and carbon monoxide to a first F-T reactor, removing a hydrocarbon stream from the first reactor, removing a first gaseous effluent stream from the first reactor, conveying a portion of the first gas effluent stream to a second F-T reactor, adding an additional source of hydrogen to the second F-T reactor, removing a hydrocarbon stream from the second reactor, and removing a second gaseous effluent stream from the second F-T reactor.

Embodiments of the apparatus for carrying out the method are also disclosed.

The synthesis gas is essentially hydrogen and carbon monoxide in embodiments, but may also include some unconverted methane and carbon dioxide.

In embodiments, the hydrocarbon streams removed from the first and/or second F-T reactor are liquid streams.

Optionally, the process includes one or more of separating water and/or CO₂ from the first gaseous effluent stream, separating water and/or CO₂ from the second gaseous effluent stream, and adding an additional source of hydrogen to the first F-T reactor. The additional source of hydrogen in the hydrocarbon stream from the second reactor is greater than the additional source of hydrogen added to the first F-T reactor.

In embodiments, the or each additional source of hydrogen is essentially pure hydrogen, however, it may include some additional inert constituents such as methane, CO₂ or nitrogen. Possibly, the or each additional source of hydrogen additionally includes CO and the H₂/CO or CO₂ ratio is greater than 2, preferably >2.5. In embodiments, at least a portion of the additional hydrogen is first produced in a steam reformer.

The process may or may not include recycling at least a portion of the dry second (last) gaseous effluent stream to the first F-T reactor, but can include recycling at least a portion of the dry first gaseous effluent stream to the first F-T reactor. The two reactors may or may not have different operating temperatures. In embodiments, the operating temperature of the first F-T reactor is in the range of about 200 to 260° C. and the operating temperature of the second F-T reactor is in the range of about 190 to 250° C. In an optional arrangement, the product streams comprise only gaseous hydrocarbons, by operating at a significantly higher temperature, up to 400° C.

A further possible optimization is to remove hydrocarbons from the gaseous effluents, e.g. by condensing at a reduced temperature. Thus, already produced valuable hydrocarbons are separated out as product, and any unnecessary recycle of these products is avoided. The gaseous stream, or a portion of this stream, may be recycled to the main syngas generator. This recycled stream may contain CO₂ or H₂O for participation in the syngas reactions (water gas shift and steam reforming).

In embodiments, the hydrogen conversion in both F-T reactors is greater than or equal to about 60%, such as about in an embodiment 65%. The total transverse cross-sectional area of the second F-T reactor is less than 50% that of the first F-T reactor in embodiments. The diameter of the second F-T reactor is less than 50% that of the first F-T reactor in embodiments. However, it can be advantageous to increase the diameter of the second reactor to approach or even surpass that of the first reactor if the second F-T reactor is common for at least two first F-T reactors, thus reducing the total number of reactors. There may be more than two F-T reactors in series. Then any reactor may relate to its preceding reactor in the series as the second reactor above is said to relate to the first.

In embodiments, the main active catalytic component in the first and/or the second reactor is cobalt. Cobalt can be impregnated into or on to any convenient catalyst carrier material, examples being alumina, titania and silica. Promoters such as platinum, rhenium or ruthenium can be added, however, any other suitable catalyst carrier and promoter(s) described in the literature can be used. The catalyst carrier can be in any convenient shape, e.g. spheres, pellets, extrudates or monoliths. Optionally, other Fischer-Tropsch catalytic metals like iron, nickel or ruthenium can be employed instead of or in addition to cobalt.

The synthesis gas is first produced from natural gas in embodiments. The syngas may be produced in an autothermal reformer, with or without pre-forming of the natural gas. The H₂/CO ratio of the gas leaving the reformer is greater than about 1.9, between about 1.90 and 1.99, in embodiments.

In embodiments, both or all the F-T reactors are of the slurry bubble column type, however, any of the reactors, may be a fixed bed, fluidised bed, or ebulating bed reactor. Other reactor configurations and catalyst deployment systems, such as a monolith, honeycomb, plate or micro-channel type, can also be employed or the reactor can be a transport reactor.

The reaction pressure is in the range of about 10-60 bar, e.g. 15 to 40 bar in embodiments. The superficial gas velocity may be in the range of about 5 to 200 cm/s, such as 20 to 50 cm/s in the case of a slurry bubble column reactor.

The hydrocarbon product or products are subsequently subjected to fractionation and post-processing, e.g. de-waxing, hydro-isomerisation, hydro-cracking and combinations of these in embodiments.

### BRIEF DESCRIPTION OF THE DRAWINGS

The invention may be carried in to practice in various ways and will now be illustrated using the following Examples and with reference to the drawings, in which:

FIG. 1 is a schematic flow diagram of a reference system with a single F-T reactor; and

FIG. 2 is a schematic flow diagram of a system according to the invention.

### DETAILED DESCRIPTION

It has been found that considerable improvements in the performance of FT-plants, in term of investment, carbon efficiency and reactor size, can be achieved individually or simultaneously by converting the syngas using at least two FT-reactors in series. In such an FT-reactor block, it is important to arrange the recycle of off-gases in an optimal way for one or several reactors, and also to optimise the FT-block tail-gas treatment and recycle to the syngas unit. It has been surprisingly discovered that adding a separate hydrogen stream to the inlet stream to the second F-T reactor and optionally also to the first FT-reactor, can address the mentioned challenges. The improved performance is even found when this hydrogen is produced by a separate means of generating hydrogen, such as a steam reformer, and all losses and emissions from such a unit are accounted for.

The effect of adding hydrogen in this way, particularly when a cobalt catalyst is used in the FT-reactors, can be understood by considering the following.

Normally an un-stoichiometric syngas, i.e. H₂/CO<2, is fed to the (first) FT-reactor to give a low H₂/CO ratio in the reactor, which promotes high C5+ selectivity. However, the consumption ratio is around 2 or slightly above. This means that hydrogen should be added if all the CO is to be converted. It has now been found that this is most efficiently accomplished by having two (or more) reactors in series, and by adding extra hydrogen to the second (and preferably any subsequent) reactor(s).

A similar advantage can be foreseen for a CTL (Coal to Liquids) or BTL (Biomass To Liquids) plant with an FT-reactor operated with a cobalt or an iron catalyst. At the outset, a CTL or BTL plant, based on gasification of coal or biomass, gives rise to a syngas with an even lower H₂/CO ratio, but potentially varying over a wide range, from below 0.5 to approaching 2 (Martyn V. Twigg (ed.), Catalyst Handbook, 2nd ed., Wolfe Publishing, 1989, p. 195). Balancing the feed composition with addition hydrogen can become even more important in these cases.

In WO0063141, a system with several Fischer-Tropsch reactors in series has been described using slurry reactors with iron catalyst. The point made is that for a Fe catalyst, the two main reactions are:

2 H₂+CO→—CH₂—+H₂O synthesis

H₂O+CO→CO₂+H₂ shift reaction

and using natural gas as feed, the hydrogen to carbon monoxide ratio in the F-T reactor normally is much higher than the consumption ratio in the reactor. This occurs because the iron catalyst has a significant shift activity, thereby consuming extra CO and producing extra hydrogen. Therefore a significant amount of CO₂ and surplus hydrogen is produced. To reduce this effect, it is proposed to use several reactors in series with removal of water in between, thereby reducing the average water vapour pressure and suppressing the shift reaction. In embodiments of the invention, it has been found advantageous to remove water between the reactors and to employ a cobalt catalyst, both to increase the partial pressures of the reactants and thereby the reaction rate, and to protect the catalyst from being partially oxidised. Otherwise the intention and solution is the opposite of that in WO 0063141. Using a feed H₂/CO ratio slightly below 2 for a cobalt catalyst, e.g. 1.8 to 1.98, means that the reactor exit ratio will be even lower, e.g. between 1 and 1.5. Therefore the limiting reactant in the FT section is hydrogen and not CO as it is for the iron-catalysed case described in WO 0063141. To compensate for this effect in the present invention, it has been found that it is advantageous to add hydrogen between the reactors, while the conversion in the FT-loop can be further increased by gas recycle around or between one or several of the reactors. No such hydrogen addition or recycle is contemplated in WO 0063141.

WO03010117 also describes a Fischer-Tropsch reaction carried out in reactors arranged in a series. Each stage in the series may consist of several reactors, e.g. 4 parallel reactors in the first stage and 2 parallel reactors in the second stage. However, no hydrogen is added between the reactors to adjust the hydrogen to CO ratio. Also, a moderate single-pass conversion of typically 53% or less is employed, compared to a preferred conversion of at least 55%, or preferably above 60%, more preferably above 65% of the limiting component in the present invention. Further, in this reference, the total syngas conversion in the FT-section for two reactors in series is in the range of about 84-90%, whereas with the present invention, it has been found that by adding hydrogen between the reactors and recycling unconverted gas around the first reactor, it is possible to increase the conversion in the FT-loop to above 90%, or even above 92% or in the most optimal arrangement, to above 94%. Here, the F-T loop is the entire F-T section of the overall plant, and is independent of the number of reactors and the internal recycle configuration in the F-T section of the plant.

Process Simulations

In accordance with various embodiments, a number of process simulations have been performed using a spread sheet model. The model also provides investment cost estimates, based on scaling of a more detailed base case simulation and cost estimate, as well as estimated carbon efficiencies and CO₂ emissions. The carbon efficiency is calculated as the carbon yield in the FT-products relative to carbon in the natural gas feed to the process, i.e. to the synthesis gas unit, and includes losses related to fuel consumption within the GTL plant and upgrading by mild hydrocracking/isomerization to give maximum diesel fuel yield. If additional hydrogen is provided to the GTL process, this is included in the carbon efficiency by adding carbon consumption by steam reforming, both for the natural gas feedstock and fuel to fired heaters.

The model comprises two basic reactor models, an ATR (AutoThermal Reformer) reactor model for the syngas generation and a Fischer-Tropsch reactor model for a slurry bubble column with a cobalt based catalyst. Process off-gases are used as fuel and supplemented with natural gas feed to cover total requirements.

The ATR model calculates the reaction products for a given feed composition at equilibrium conditions and fixed reactor outlet temperature and pressure. Ideal gas conditions are assumed and the reactor inlet temperature is estimated from the heat balance by assuming adiabatic reactor conditions.

The FT model is based on reaction kinetics for a set of characteristic reactions. The following reactions with corresponding reaction rates are included in the model:

(1) C1:

3H₂+CO═CH₄+H₂O,

(2) C2-C4:

7H₂+3CO═C₃H₈+3H₂O,

(3) C5+:

(2n+η)H₂+nCO ═CnH_{2n+2η}+nH₂O

(4) Shift:

CO+H₂O═H₂+CO₂

where n is the carbon number for the FT product and η is the fraction of saturated components in the product (η=0 means 100% mono olefins). The C5+ product distribution is predicted by a Schultz-Flory distribution. The mean carbon number is then calculated from the Schultz-Flory distribution and the α-value. The olefins content in the product is estimated as percent mono olefins in the C5+ product. All other hydrocarbon components are assumed to be alkanes. The reactor size is estimated by scaling a reference reactor design. The diameter is scaled on the basis of constant superficial gas velocity, while reactor height is calculated relative to catalyst load.

The basic flow sheet model input variables are natural gas feed rate [Sm3/hr], hydrogen feed to synthesis gas unit [Sm3/hr], oxygen feed rate to the ATR, ATR outlet temperature and pressure, optional hydrogen make-up to Fischer-Tropsch synthesis loop [Sm3/hr], steam-carbon ratio in the ATR feed, Fischer-Tropsch loop purge [as % of gas product], tail gas recycle ratio from FT unit to synthesis gas unit [as % of loop purge].

It will be appreciated that the invention is not restricted to specific reactor types or designs. For example, the syngas unit can be any type or combination of ATR, steam reforming, catalytic partial oxidation, partial oxidation, heat exchange reformer, convective reformer, compact reformer etc. A pre-reformer may be included if it is found desirable. The FT-reactor can be of any type and design like a slurry bubble column, fixed-bed, fluidized-bed, transport reactor, ebulating bed, monolith type, compact heat-exchanger type etc. Further, the FT-products can be upgraded to final products like diesel fuel, lubricant base oil, alfa-olefins etc. in any way known in the art. Any known FT-catalyst can be employed, e.g. based on cobalt or iron as the main catalytic component, with promoters like rhenium, platinum or ruthenium, and supports like alumina, silica, titania or other inorganic porous oxides.

All examples are based on the common assumptions of a fixed reactor outlet temperature and pressure in the ATR (1000° C.; 37 bar) and FT reactors (228° C.; 30.1 bara), fixed conditions in synthesis gas unit for steam/carbon ratio, oxygen feed rate, hydrogen feed rate upstream pre-reformer and adiabatic temperature rise in the ATR. Further, 60% hydrogen conversion per reactor stage in the FT unit has been assumed. The additional parameter that is adjusted is the hydrogen make-up to the FT-reactor(s).

Single F-T Reactor

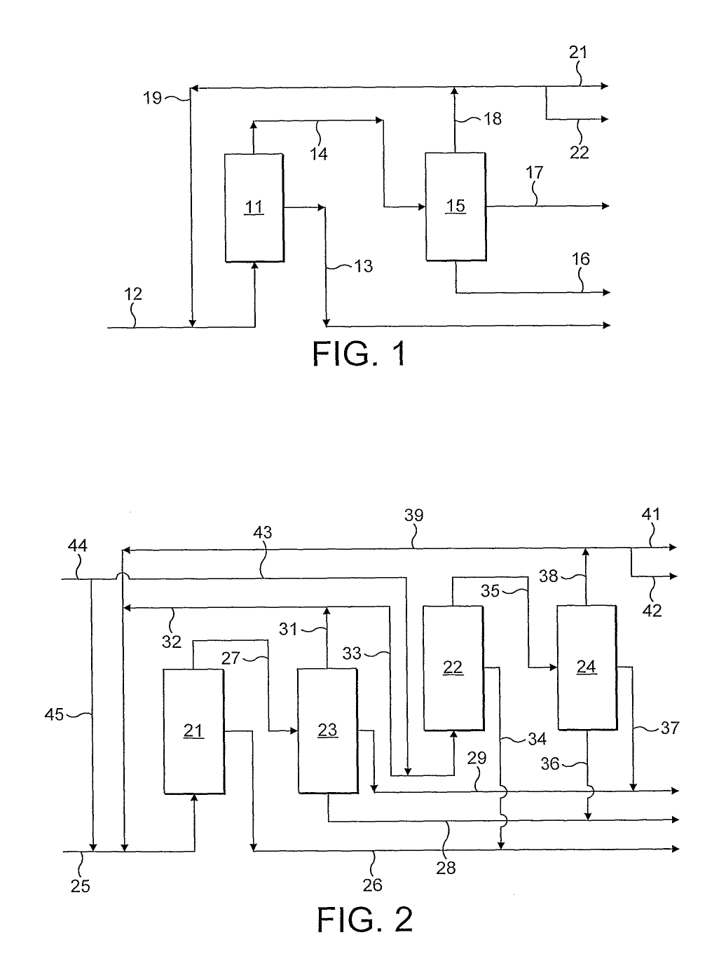

In the system in FIG. 1, synthesis gas is fed to an F-T reactor 11 via a syngas feed stream 12. From the reactor 11 there is an F-T wax product stream 13, and an F-T gas stream 14. The gas stream 14 is fed to a separator (or separator system) 15 where water is removed via a water stream 16 and F-T liquid product is removed via a liquid stream 17. Tail gas containing hydrogen is removed via a tail gas stream 18 and a portion 19 is recycled to the reactor 11. The remainder is purged 21 and/or recycled 22 to the syngas generator.

### Example 1

### Reference Case

A reference case has been modeled and simulated for a world scale GTL plant of 60,000 bbl/day. Such a plant can conveniently have 4 parallel processing lines. The reference case includes a synthesis gas unit comprising pre-reforming with moderate upstream hydrogen feed (2.2 tons/hr), oxygen feed from an air separation unit (4×3,600 tons/day), autothermal reforming with a feed furnace, auxiliary hydrogen generated by a separate steam reformer, and a waste heat recovery unit. The Fischer-Tropsch unit is as shown in FIG. 1 and features a single stage reactor with reactor recycle tail gas recycle to synthesis gas unit upstream pre-reformer, and purge gas to fuel. Further, the parameters are tuned to give 90% conversion of hydrogen in the FT-block (FT-loop conversion) and a H₂/CO ratio of 1.26 leaving the reactor. The results are summarised in Table 1.

### Example 2

### Increased Loop Conversion

The system used is as shown in FIG. 1, but in this case, the FT-loop conversion of hydrogen is increased from 90 to 95% simply by reducing the purge and increasing the recycle in the FT-loop. The H₂/CO ratio in the synthesis reactor is kept constant by the added feature of hydrogen make-up from a steam reformer unit. Results are shown in Table 1.

It can be seen that the carbon efficiency can be increased 1.8% points this way, giving more product, but at the expense of a higher specific investment and a significant increase in reactor diameter, possibly beyond what is viable. It can be seen from the reduced water vapor pressure that there is a build-up of the inert concentrations in the reactors.

### Example 3

### Hydrogen Make-up

Again, the basic system used is that shown in FIG. 1 but with the addition of hydrogen make-up. This can be added as a separate stream, e.g. to line 19. Compared to the reference case in Ex. 1, hydrogen is added to the FT-loop with the consequence that the H₂/CO ratio increases. Minimal effects are seen in natural gas consumption, reactor dimensions, carbon efficiency, product yield or investment pr. barrel product. Results are shown in Table 1.

Two F-T Reactors in Series

In the system in FIG. 2, there are two F-T reactors 21, 22 and two separators (or separator systems) 23, 24. The system operates as follows.

Syngas feed is fed to the first F-T reactor 21 via stream 25. From the reactor 21 there is an F-T wax product stream 26 and an F-T gas stream 27. The gas stream 27 is fed to the first separator 23, where water is removed via stream 28 and F-T liquid product is removed via stream 29. Tail gas leaves the separator 23 via stream 31 and a portion is recycled to the first reactor via stream 32 while the remainder constitutes a feed stream 33 to the second reactor 22.

From the second reactor, there is an F-T wax product stream 34 and an F-T gas stream 35. The gas stream 35 is fed to the second separator 24, where water is removed via stream 36, and F-T liquid product is removed via stream 37. Tail gas leaves the separator 24 via stream 38 and can be recycled to the first reactor via stream 39. The remainder is purged 41 and/or recycled 42 to the syngas generator.

A hydrogen make-up stream 43 from a hydrogen source 44 can, in accordance with the invention, be fed to the second reactor 22, and optionally, via stream 45 to the first reactor 21.

Where hydrogen is added, the hydrogen can come from any suitable source, including any stand-alone hydrogen generator. Such a stand-alone hydrogen generator can be steam-reforming followed by shift reactors and PSA (pressure swing adsorption) or membrane separation. The hydrogen can also be produced by any other means such as employing alternative reformer technologies, including a heat-exchange reformer, convective reformer or compact reformer, or any sort of partial oxidation or catalytic partial oxidation. These technologies also can be used alone or in combination for the primary syngas generation in the GTL plant. Thus, as an example, if ATR is employed for the syngas production and there is spare capacity, a slip stream can be used to make the essentially pure hydrogen needed for the hydrogen make-up.

The hydrogen can also be imported from a nearby plant, e.g. a steam cracker or dehydrogenation unit, or a chlorine-alkali electrolysis unit. These chemical plants produce hydrogen as a by-product that is normally used as fuel. It is also known that hydrogen production is being considered by gasification of biomass and electrolysis of water as well as other novel techniques, e.g. photo-catalytic decomposition of water and bio-mimic processes.

It is important to realise that the effects described in the examples when hydrogen is added to one or several reactors, to a large degree can be obtained also when the hydrogen is not pure hydrogen. It can contain inert components to the FT-reaction, including some CO₂ that subsequently is recycled in part to the syngas generator, and even CO as long as the H₂/CO ratio is higher than the main syngas feed to the reactor. An attractive solution can be to use a steam reformer with shift, but omitting the hydrogen separation unit. The produced gas then has a nominal composition of 4 parts hydrogen and 1 part CO₂. This gives a nominal so-called stoichiometric number, SN═(P(H₂)−P(CO₂))/(P(CO)+P(CO₂)), of SN=3. Because SN>2, this means that even when the CO₂ is recycled to the syngas unit, excess hydrogen is added to the plant, and the full effect on the FT-reactor performance is maintained, except for some reduction in partial pressures of the reactants.

### Example 4

### Two Stage FT-Reactor Concept

A block diagram for the FT-section with 2 reactors in series is shown in FIG. 2. The variables in the simulations include the 1^{st} stage recycle as % of the gas from the 1^{st} product separator, recycle from 2^{d} product separator back to the 1^{st} FT-stage, and tail-gas recycle to the syngas unit, as well as individual hydrogen make-up to the 1^{st} and 2^{d} FT-reactor stage. It was noted that additional recycle for the 2^{nd} FT-stage has minimal effect on the simulated result. To avoid excessive water pressure in the second stage, water is removed in the first separator.

In this case no hydrogen is added to either of the FT-stages. Still the carbon efficiency increases, but this requires the use of very tall reactors. Part of the reason is that the H₂/CO ratio will be very low in the second reactor due to the very under-stoichiometric feed from the first reactor. Further, the partial pressure of water in the first reactor reaches a level where it might adversely influence the catalyst performance in terms of stability and possibly selectivity. The results are summarized in Table 1.

TABLE 1
Process simulations for a GTL plant

| | | Ex. 2 | Ex. 3 | Ex. 4 | Ex. 5 | |
|---|---|---|---|---|---|---|
| | Ex. 1 | 95% loop | Hydrogen | No H₂ | H₂ to 2^{nd} | Ex. 6 |
| | Reference | conversion | make-up | make-up | stage | Combined |
| No. of FT-reactors in | 1 | 1 | 1 | 2 | 2 | 2 |
| each train | | | | | | |
| NG feed to pre-reformer | 100.0 | 100.2 | 99.7 | 96.5 | 99.6 | 95.6 |
| (%) | | | | | | |
| FT product yield (%) | 100.0 | 105.6 | 99.8 | 98.9 | 107.6 | 107.5 |
| FT-loop conversion | 90.0 | 95.0 | 90.0 | 89.7 | 95.5 | 93.3 |
| (% H₂) | | | | | | |
| FT-H₂ make-up (Sm3/h) | 0 | 10.900 | 11.800 | 0 | 0 | 11.823 |
| 1. reactor | | | | | | |
| FT-H₂ make-up (Sm3/h) | — | — | — | 0 | 10.115 | 13.257 |
| 2. reactor | | | | | | |
| H₂/CO FT-reactor 1 | 1.26 | 1.26 | 1.36 | 1.26 | 1.26 | 1.32 |
| effluent (mol/mol) | | | | | | |
| H₂/CO FT-reactor 2 | — | — | — | 0.80 | 1.27 | 1.27 |
| effluent (mol/mol) | | | | | | |
| H₂O FT-reactor 1 partial | 4.1 | 2.7 | 4.2 | 5.8 | 3.8 | 4.5 |
| pressure (bara) | | | | | | |
| H₂O FT-reactor 2 partial | — | — | — | 3.7 | 2.5 | 3.1 |
| pressure (bara) | | | | | | |
| Reactor diameter (m) | 10.2 | 12.4 | 10.2 | 8.1 | 10.5 | 9.6 |
| 1. reactor | | | | | | |
| Reactor diameter (m) | — | — | — | 4.4 | 3.5 | 3.9 |
| 2. reactor | | | | | | |
| Reactor height (m) | 24.4 | 20.8 | 24.1 | 27.6 | 23.6 | 24.9 |
| 1. reactor | | | | | | |
| Reactor height (m) | — | — | — | 30.9 | 21.8 | 23.6 |
| 2. reactor | | | | | | |
| Carbon efficiency (%) | 68.5 | 71.3 | 68.2 | 70.0 | 74.4 | 75.4 |
| Investment pr. barrel | 100.0 | 101.5 | 100.0 | 100.0 | 96.7 | 96.7 |
| liquid product | | | | | | |

### Example 5

### Hydrogen Make-up to Second FT-Reactor

The FT process layout is as for the two-reactor case in Ex. 4, i.e. FIG. 2, but make-up hydrogen is added to the second reactor so that the H₂/CO ratio is about the same for both reactors. It can now be seen that the water vapor partial pressure is moderate in both reactors and that the maximum reactor dimensions are comparable to the reference case. A huge benefit can be seen for the carbon efficiency, up from 68.5 to 74.4%, accompanied by a similar enhancement in the product yield. Simultaneously, the investment is reduced by 3.3% points.

### Example 6

### Hydrogen Make-up to Both FT-Reactors

This is an optimization of Ex. 5 by also adding hydrogen to the first FT-reactor, thereby increasing the carbon efficiency further to 75.4%. It is also significant that the maximum reactor diameter is reduced by 60 cm giving a more comfortable size. Alternatively this can give room for added train capacity by 13%, assuming that it is no longer the ASU that is limiting.

From the previous examples it is clear that two FT-reactors in series with hydrogen make-up have a number of advantages. One striking point is that the second reactor has a very moderate diameter and therefore a plant lay-out is feasible where the second reactor is common for all four process trains (or for two), increasing the diameter of the second reactor to the range of about 5-7.4 m, which still is moderate. If a tail-gas reformer is selected, this can be added after the common second FT-reactor.

From the above, it can also be expected that further improvements in carbon efficiency, product yield and cost savings can be achieved by adding a third FT-reactor in series, or even more. From the above it is clear that any combination of reactors in series and in parallel can be used with appropriate and optimized make-up of hydrogen to some or all of the reactors. It is advantageous, however, that in a series of reactors there are fewer reactors in parallel as one goes from one stage to the next. As an example, 9 parallel reactors can be used in the first stage of a series, 3 in parallel in the next and one 1 reactor in the last stage. Optimisation will also include the process conditions, e.g. it is possible to vary the temperature individually for each reactor.

1-35. (canceled)

36. A process for converting synthesis gas comprising hydrogen and carbon monoxide into hydrocarbons using a Fischer-Tropsch (F-T) synthesis reaction comprising:
conveying a gas feed of hydrogen and carbon monoxide to a first F-T reactor;
removing a hydrocarbon stream from the first F-T reactor;
removing a first gaseous effluent stream from the first F-T reactor;
conveying a portion of the first gas effluent stream to a second F-T reactor;
adding an additional source of hydrogen to the second F-T reactor;
removing a hydrocarbon stream from the second F-T reactor; and
removing a second gaseous effluent stream from the second F-T reactor.

37. A process according to claim 36, further comprising separating water from the first gaseous effluent stream.

38. A process according to claim 36, further comprising separating water from the second gaseous effluent stream.

39. A process according to claim 36, further comprising adding an additional source of hydrogen to the first F-T reactor.

40. A process according to claim 36, wherein the additional source of hydrogen to the first F-T reactor is an external source.

41. A process according to claim 36, wherein the additional source of hydrogen to the second F-T reactor is an external source.

42. A process according to claim 39, wherein the additional source of hydrogen to the second F-T reactor is equal to or greater than the additional source of hydrogen to the first F-T reactor.

43. A process according claim 39, wherein each additional source of hydrogen is essentially pure hydrogen.

44. A process according to claim 39, wherein each additional source of hydrogen additionally includes inert constituents.

45. A process according to claim 39, wherein each additional source of hydrogen additionally includes CO, and a stoichiometric number (SN) and an H₂/CO ratio are both greater than 2.

46. A process according to claim 36, wherein at least a portion of the additional hydrogen to the first and second F-T reactors is first produced in a steam reformer.

47. A process according to claim 37, further comprising recycling at least a portion of the dry first gaseous effluent stream after water removal to the first F-T reactor.

48. A process according to claim 38, further comprising recycling at least a portion of the second gaseous effluent stream to the first F-T reactor.

49. A process according to claim 36, wherein the first and second F-T reactors have different operating temperatures.

50. A process according to claim 49, wherein the operating temperature of the first F-T reactor is in a range of 200° to 260° C. and the operating temperature of the second F-T reactor is in a range of 190° to 250° C.

51. A process according to claim 36, wherein the partial pressure of water in the first F-T reactor is greater than a partial pressure of water in the second F-T reactor.

52. A process according to claim 51, wherein the partial pressure of water in the first reactor is below 6 bara and the partial pressure of water in the second F-T reactor is below 4 bara.

53. A process according to claim 36, wherein hydrogen conversion in both the first and second F-T reactors is ≧60%.

54. A process according to claim 53, wherein the hydrogen conversion in both F-T reactors is between 65% and 80%.

55. A process according to claim 36, wherein a total transverse cross-sectional area of the second F-T reactor is less than 50% of a total transverse cross-sectional area of the first F-T reactor.

56. A process according to claim 55, wherein a diameter of the second F-T reactor is less than 50% of a diameter of the first F-T reactor.

57. A process according to claim 36, wherein an FT loop conversion is larger than 90%.

58. A process according to claim 57, wherein the conversion is between 92% and 98%.

59. A process according to claim 36, wherein the second F-T reactor is common for at least two first F-T reactors.

60. A process according to claim 36, comprising more than two F-T reactors in series.

61. A process according to claim 36, wherein a main active catalytic component in the first and the second reactor is cobalt.

62. A process according to claim 36, wherein a synthesis gas is first produced from natural gas.

63. A process according to claim 62, wherein a synthesis gas is produced in an autothermal reformer, with or without pre-reforming of the natural gas.

64. A process according to claim 63, wherein an H₂/CO ratio of the gas leaving the reformer is >1.9 and <2.0.

65. A process according to claim 36, wherein each F-T reactor is a three-phase slurry bubble column reactor.

66. A process according to claim 5, wherein an F-T reaction pressure in each F-T reactor is in the range of 10-60 bara.

67. A process according to claim 66, wherein the reaction pressure is in the range of 15 to 40 bara.

68. A process according to claim 65, wherein a superficial gas velocity in the first and second F-T reactors in the range of 5 to 60 cm/s.

69. A process according to claim 68, wherein the superficial gas velocity is in the range of 20 to 50 cm/s.

70. A process according to claim 36, wherein a product of the Fischer-Tropsch synthesis reaction is subsequently subjected to post-processing.

71. A process according to claim 70, wherein the post-processing is selected from the group consisting of de-waxing, hydro-isomerisation, hydro-cracking and combinations thereof.
